(19) United States
(12) Patent Application Publication
(10) Pub. No.: US 2012/0308614 A1
(43) Pub. Date: Dec. 6, 2012

(54) COMPOSITION FOR THE CONTROLLED RELEASE OF BUPRENORPHINE

(75) Inventors: Aline Moulin, Lyon (FR); You-Ping Chan, Ternay (FR)

(73) Assignee: Flamel Technologies, Venissieux (FR)

(21) Appl. No.: 13/483,820

(22) Filed: May 30, 2012

Related U.S. Application Data
(60) Provisional application No. 61/491,614, filed on May 31, 2011.

(30) Foreign Application Priority Data
May 30, 2011 (FR) FR 11 54686
May 30, 2012 (IB) PCT/IB2012/052689

(51) Int. Cl. A61K 31/485; A61P 25/04; A61K 9/14; B82Y 5/00

(52) U.S. Cl. 424/400; 514/279; 977/773; 977/915; 977/775; 977/788

(57) ABSTRACT

The present invention relates to a novel aqueous liquid pharmaceutical composition for the controlled release of buprenorphine or of an analogue of buprenorphine, comprising at least one prodrug with low aqueous solubility of said buprenorphine or analogue of buprenorphine, and at least one polymer having a linear backbone chosen from the polyglutamates, polyaspartates, poly(meth)acrylates and polysaccharides, to which one or more hydrophobic groups are grafted.

This application claims the benefit of each of U.S. Provisional Patent Application No. 61/491,614 filed May 31, 2011, French Patent Application No. FR 11 54686 filed May 30, 2011 and International Patent Application No. PCT/IB2012/052689 filed on May 30, 2012, each of which is incorporated by reference herein in their entirety.

The invention relates to pharmaceutical compositions dedicated to the controlled release of buprenorphine or an analogue of buprenorphine, an active ingredient which is useful in the treatment of pain. More particularly, it describes compositions comprising a prodrug of buprenorphine or an analogue of buprenorphine and a specific polymer. These compositions advantageously allow the duration of action of buprenorphine to be sustained while having a rapid action just after administration.

Buprenorphine is a medicament used as an analgesic and in the replacement therapy of dependence on opiates. It is a partial agonist and an antagonist of the opioid receptors. Today it is marketed in various forms, as a transdermal patch, as a sublingual lozenge or for injectable administration by intramuscular or intravenous route.

The use of patches or lozenges is generally favoured in the treatment of chronic pain. Thus, certain patches have the ability to procure a release over a week. However, these presentations do not prove to be suitable for use in animals. Moreover, as regards more particularly patches, they are not appropriate for procuring a rapid analgesic effect after application. Finally, the presentations for oral or transdermal administration require higher doses to compensate for the loss of bioavailability. For example, it is noted, in the case of the application of a transdermal patch, that the release of buprenorphine reaches the stationary state after three days and that the bioavailability is of the order of only 15%. By oral route, the bioavailability is only 10%.

The injectable form, used in particular by intramuscular or intravenous route in humans and animals, is generally favoured in the context of the treatment of pain in pre- or post-operative surgery. By way of illustration of these injectable forms there can in particular be mentioned that marketed under the trade name Buprenex® or Buprecare®, which is an aqueous solution of buprenorphine in its hydrochloride salt form at 0.3 mg/mL. The dose to be administered to a dog is 0.1 to 0.2 mg every 8 hours and to humans 0.2 to 0.6 mg every 8 hours. It is much used in the veterinary field for the treatment of pain. However, to be effective over several days, the administrations must be frequent and can lead to side effects linked to concentration peaks which occur too frequently.

A formulation for an administration by parenteral route, offering both a rapid start of action and a duration of action of several days, would be useful for several reasons. It would make it possible to reduce the number of doses, limit or even overcome the side effects, and therefore better treat the pain.

The slow-release formulations of buprenorphine currently proposed include hydrophobic polymers or oil-in-water emulsions.

Thus, the Patent Application US 2008/0227805 describes oil-in-water emulsions of buprenorphine at different pHs. This type of formulation makes it possible to effectively regulate the rate of release but the total duration of action remains limited and does not exceed, for example, 30 hours in the rat.

A micro-particulate formulation of buprenorphine, based on a lactide and glycolide copolymer (PLGA) has also been developed, then tested in humans (Sigmon et al. Addiction 2006, 101, 420-432) in the context of the treatment of dependence. However, this approach is also not totally satisfactory. The clinical study reveals a pharmacokinetic profile extended up to 5 weeks but the maximum plasma concentration (Cₘₐₓ) is only reached after 2 or 3 days, and the variability is relatively significant.

As for it, the Patent Application WO 2010/009451 describes microparticles based on poly-ortho ester and buprenorphine having a duration of action in the dog of 3 to 8 days and the maximum plasma concentration (Cₘₐₓ) is obtained after at least 10 hours.

Another approach consists of using buprenorphine in the form of a prodrug. Buprenorphine is then generated in situ in particular via the hydrolysis of a hydrolysable bond. This approach has several advantages.

Thus, Stinchcomb et al. (Pharm. Res. 1996, 13, 1519-1523) and Imoto et al. (Biol. Pharm. Bull. 1996, 263-267) describe C₁ to C₄ alkyl esters of buprenorphine and study their absorption by transcutaneous route. A reduction of the melting point and an increase in the solubility in an oily phase is noted. Its transcutaneous passage is improved.

The U.S. Pat. No. 7,084,150 describes prodrugs of buprenorphine deriving from the functionalization of buprenorphine with various hydrophilic or hydrophobic ester groups for a use in particular by transdermal route. No example of an application is given.

The Patent Application US 2008/0076789 describes prodrugs of buprenorphine deriving from the functionalization of buprenorphine with hydrophilic groups making it possible to facilitate transdermal passage.

The Patent Application WO 2007/110636 describes succinate and adipate derivatives of buprenorphine, which are useful in view of their improved bioavailability by oral route.

As regards Patent Application US 2005/0075361 by Wang et al., it describes injectable compositions based on a prodrug of buprenorphine (mono- or di-carboxylic ester) solubilized in an oily phase. These compositions, which are all injected by intramuscular route, make it possible to obtain durations of action in the rat comprised between 50 and 96 hours.

In parallel, and in an effort in particular to improve the bioavailability by oral route or the transdermal passage of buprenorphine, to slow down its release or also to prevent its misuse, the formulation of prodrugs of buprenorphine in the nano- or microparticle state has been proposed.

Thus, Wang et al. (Eur. J. Pharm Sci. 2009, 38, 138-146) describe formulations dedicated to an administration by intravenous route and comprising lipidic nanoparticles incorporating respectively prodrugs of buprenorphine of C₃ to C₇ ester type. The associated analgesic effect has been observed in the rat for up to 10 hours. This system has also been evaluated in the case of transcutaneous permeation in the mouse (Wang et al., J. Microencapsulation 2009, 26, 734-747). The results show very low transport kinetics compared with the formulae using unmodified buprenorphine.

Consequently, it is apparent from the above that the use of a hydrophobic prodrug of buprenorphine solubilized in an oil or conveyed in lipidic nanoparticles is the pharmaceutical formulation conventionally proposed in order to obtain a slow release of buprenorphine via an administration by intramuscular route.

However, the injection of these types of formulations requires relatively broad needles, from 18 to 21 gauge, due to the high viscosity. Now, it is well known that injection by intramuscular route often leads to strong and persistent pain and requires intervention by health professionals. Multiple intramuscular injections can also cause abscesses or fibroses (Gribaldi's Drug Delivery Systems in Pharmaceutical Care Desai and Lee, 2007).

The present invention aims in particular to overcome these drawbacks.

More precisely, the inventors have found, against all expectations, that a liquid composition of low viscosity, comprising a prodrug of buprenorphine or one of its analogues and a specific polymer, makes it possible to obtain a duration of action of several days and proves to be compatible with an administration by subcutaneous route.

Thus according to one of its aspects, the present invention relates to an aqueous liquid pharmaceutical composition, for the controlled release of buprenorphine or an analogue of buprenorphine, comprising at least one prodrug with low aqueous solubility of said buprenorphine or of an analogue of said buprenorphine and at least one polymer having a linear backbone chosen from the polyglutamates, polyaspartates, poly(meth)acrylates and polysaccharides, to which one or more hydrophobic groups are grafted.

According to the invention, the polymer and the prodrug of buprenorphine or an analogue of buprenorphine are associated in a non-covalent manner.

By “pharmaceutical”, is meant a composition intended for treating humans or animals.

Within the context of the present invention, the prodrug with low aqueous solubility of buprenorphine or of one of its analogues has a solubility, measured at room temperature, of less than 1 mg/ml in water at pH 7.

It should be noted that an aqueous liquid composition according to the invention can be obtained by the addition of an aqueous liquid, in particular water, to a solid composition, in particular in the form of a powder, said solid composition comprising at least one prodrug with low aqueous solubility of said buprenorphine or of an analogue of said buprenorphine and at least one polymer having a linear backbone chosen from the polyglutamates, polyaspartates, poly(meth)acrylates and polysaccharides, to which one or more hydrophobic groups are grafted. This solid composition constitutes another aspect of the invention.

Such a solid composition can itself be formed beforehand by dehydration, for example by lyophilization, of an aqueous liquid composition according to the invention.

According to another of its aspects, the present invention comprises a treatment method for a human or an animal, comprising the administration by subcutaneous route of a composition according to the invention as defined previously.

Other characteristics, advantages and embodiments of compositions according to the invention will become more apparent on reading the description which follows.

In the remainder of the text, the expressions “comprised between . . . and . . . ”, “ranging from . . . to . . . ” and “varying from . . . to . . . ” are equivalent and are meant to signify that the limits are inclusive, unless otherwise specified.

Unless otherwise indicated, the expressions “containing a” and “comprising a” should be understood as “containing at least one” and “comprising at least one”.

Within the meaning of the invention, the term “approximately” means that the value which follows this term is verified taking account of the limits of experimental error acceptable to a person skilled in the art.

Composition According to the Invention:

Preferably, an aqueous liquid composition according to the invention has a viscosity, measured at 20° C. and at a shear rate of 10 s⁻¹, of less than 200 mPa·s, preferably comprised between 2 and 100 mPa·s.

The viscosity can be measured at 20° C., using standard equipment such as for example an imposed stress rheometer (Gemini, Bohlin) on which a cone-plate type geometry is installed (diameter of 4 cm and angle of 1°).

Moreover, the compositions of the invention are advantageously sterile.

The word “sterile” is meant to describe an environment capable of guaranteeing a compound or composition which contains it the harmlessness required for a subcutaneous administration. In particular, it is essential that the composition formed of an aqueous liquid, of the prodrug with low solubility of buprenorphine or an analogue of buprenorphine and of said polymer of the invention, and needing to be administered according to an injection technique, be devoid of any contaminant capable of starting an undesirable secondary reaction in the host organism.

A composition according to the invention can be easily made sterile during its preparation by filtration through a 0.2 μm filter.

Prodrug of Buprenorphine or an Analogue of Buprenorphine

According to one of its aspects, a composition of the invention comprises at least one prodrug with low aqueous solubility of buprenorphine (Bu) or of one of its analogues.

Within the meaning of the invention, a prodrug of buprenorphine advantageously has the structure (I) below:

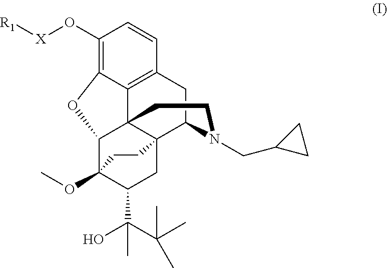

in which R is chosen from the groups:
linear C₂ to C₂₀ alkyls, branched C₃ to C₂₀ alkyls optionally comprising at least one unsaturation,
C₃ to C₆ cyclic alkyls; and
substituted phenyl and phenalkyls;

and X is a C═O, O—C═O or NH—C═O group, thus forming an ester, carbonate or carbamate bond, respectively.

Preferably, the R group is chosen from the ethyl, propyl, iso-propyl, butyl, iso-butyl, sec-butyl, hexyl, 2-ethylhexyl, cyclohexyl, heptyl, octyl, dodecyl, tridecyl, tetradecyl, pentadecyl, hexadecyl, heptadecyl, octadecyl, phenyl and benzyl groups.

As specified above, a composition according to the invention can also contain a prodrug of an analogue of buprenorphine as active ingredient.

Within the meaning of the present invention, by “analogues” of buprenorphine is meant the substituted forms of buprenorphine, subject to these substitutions not being detrimental to its therapeutic activity and it being understood that the phenol group of buprenorphine remains accessible for forming a prodrug.

More particularly, a prodrug of an analogue of buprenorphine has by analogy to the prodrugs of buprenorphine covered by formula (I) presented above, the R—X—O-phenyl unit.

In particular, it proves possible, according to the present invention, to use the analogues of buprenorphine described in the U.S. Pat. No. 5,849,915, the Patent Application WO 2010/014229 and in the article by Taline et al. (Journal of Pharmacology and Experimental Therapeutics (2011), 336(3), 952-961).

The prodrugs of analogues of buprenorphine can thus be the substituted forms of structure (I) above.

More generally, the prodrugs of buprenorphine and prodrugs of analogues of buprenorphine can be mentioned of which the general chemical formula comprises at least the following structural unit:

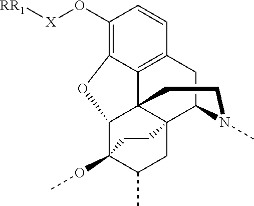

with R and X being as defined previously and the bonds in dotted lines showing the potential substitution sites.

The prodrugs considered according to the invention can be synthesized easily according to operating methods adapted from the literature. For example, the derivatives of ester type can be obtained according to operating methods described in the literature: Stinchcomb et al. (Pharmaceutical Res. 1995, 12, 1526-1529), Que et al. (Acta. Anaes. Taiwanica. 2005, 43, 11-16), by implementing, for example, the reaction shown below.

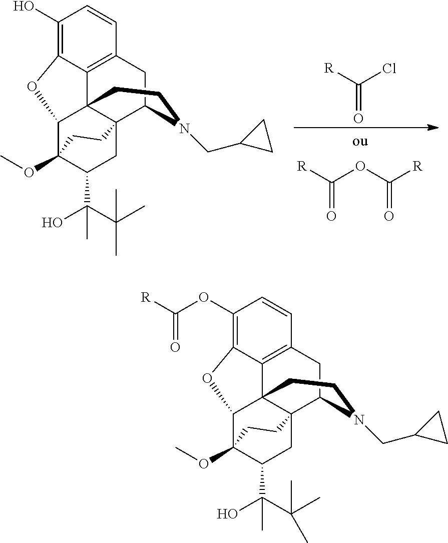

The derivatives of carbonate or carbamate type can be obtained according to the operating methods described for example in the Patent Application US 2008/0076789.

Preferably, the prodrug is a prodrug of buprenorphine ester type, and in particular corresponds to the previous formula (I) in which the X group is a C═O group.

In particular, the prodrug of buprenorphine can be of the previous structure (I), in which R is chosen from the linear C₂ to C₂₀ alkyls and the branched C₃ to C₂₀ alkyls and X is a C═O group.

According to a particular embodiment, a composition of the invention uses a prodrug of buprenorphine chosen from buprenorphine 6-isobutyrate (BuC4), buprenorphine 6-enanthate (BuC7) and buprenorphine 6-myristate (BuC14), and in particular buprenorphine 6-isobutyrate or buprenorphine 6-enanthate.

According to a particular embodiment, an aqueous liquid composition according to the invention comprises a content of prodrug(s) of buprenorphine or of prodrug(s) of an analogue of buprenorphine, ranging from 0.5 to 10 mg/mL, preferably from 1 to 5 mg/mL.

Polymer According to the Invention

According to another of its aspects, a composition of the invention comprises, as well as the prodrug of buprenorphine or an analogue of buprenorphine, at least one specific polymer.

Linear Backbone

The polymer considered according to the invention has a linear backbone chosen from:

poly(glutamic acid) (of alpha or gamma type), poly(aspartic acid) (of alpha or alpha/beta type),

poly(acrylic acid) or poly(methacrylic acid), and

the polysaccharides such as dextran or one of its derivatives such as in particular carboxymethyl dextran or hydroxyethyl dextran or pullulans.

According to a particular embodiment, the linear backbone of the polymer of the invention is chosen from poly(glutamic acid) or poly(aspartic acid).

It is understood that the residual carboxylic functions of the poly(glutamic acid), poly(aspartic acid) and poly(acrylic acid) grafted according to the invention, can occur in a form either neutral (COOH form) or ionized (COO⁻ anion), according to the pH and the composition. The polymer neutrality is provided by a counter-cation that can be a monovalent metal cation, preferably a sodium or potassium ion.

Therefore the terms i) polyglutamate or poly(glutamic acid), ii) polyaspartate or poly(aspartic acid) and iii) polyacrylate or poly(acrylic acid) will be used interchangeably.

Hydrophobic Groups

The polymer of the invention is grafted with one or more hydrophobic groups.

The pendant hydrophobic groups can be more particularly chosen from the linear C₂ to C₂₀ alkyls, the branched C₃ to C₂₀ alkyls, the hydrophobic amino acids bound by their nitrogen atom, cholesterol and tocopherol.

The hydrophobic groups which are quite particularly suitable can be chosen from the following radicals: octyloxy-, dodecyloxy-, tetradecyloxy-, hexadecyloxy-, octadecyloxy-, 9-octadecenyloxy-, tocopheryl- and cholesteryl-, preferably alpha-tocopheryl-; the hydrophobic amino acids such as leucine, valine, phenylalanine, tryptophan or tyrosine or one of their derivatives; octylamino-, dodecylamino-, tetradecylamino-, hexadecylamino- and octadecylamino.

According to an advantageous variant of the invention, the polymer of the invention is chosen from the polyaspartates or polyglutamates comprising pendant hydrophobic grafts. By way of references, known polymers which can be used according to the invention are described. In particular, reference can be made to the Applicant's documents WO 03/104303, WO 2006/079614, WO 2008/135563 and WO 2010/076519. Also, Akagi et al. (Biomacromolecules 2006, 7, 297-303) describe gamma-polyglutamic acids comprising a phenylalanine group or Kang et al. (Langmuir 2001, 17, 7501-7506) describe polyaspartic acids comprising hydrophobic linear C₁₂ to C₁₈ alkyl groups.

According to a preferred embodiment variant, the linear backbone is constituted by a homopolymer of alpha-L-glutamate or of alpha-L-glutamic or of gamma-L-glutamic acid.

According to another embodiment variant, the linear backbone is constituted by a homopolymer of alpha-L-aspartate or of alpha-D,L-alpha-beta aspartic acid.

Such polyamino acids are in particular described in documents WO 03/104303, WO 2006/079614 and WO 2008/135563, the contents of which are incorporated by way of reference. These polyamino acids can also be of the type of those described in patent application WO 00/30618.

A certain number of polymers which can be considered as backbone in order to form the grafted polymers according to the invention, for example of poly(alpha-L-glutamic acid), poly(alpha-D-glutamic acid), poly(alpha-D,L-glutamate) and poly(gamma-L-glutamic acid) type of variable masses are commercially available. They are then functionalized by reaction with a hydrophobic group having an alcohol or amine function in order to form the polymer of the invention via an ester or amide bond.

In the family of polysaccharides, a polymer of dextran type grafted with a lauroyl group can be obtained by reacting the dextran polymer with the acid chloride of lauric acid and the acid chloride of N-acetylmethionine in N-methylpyrrolidone. The operating method for obtaining dextran grafted with the lauroyl group is described in the U.S. Pat. No. 5,750,678 (Example 1). Polysaccharides containing hydrophobic C₁₂ to C₅₀ grafts, in particular a pullulan containing cholesterol as graft can be obtained according to the operating method described in U.S. Pat. No. 6,566,516.

According to a particularly preferred embodiment, the polymer according to the invention has a molar grafting rate with pendant hydrophobic groups ranging from 2 to 30%.

By “molar grafting rate” with hydrophobic groups, is meant the ratio of the average number of monomers bearing pendant hydrophobic groups to the total number of monomers constituting the linear backbone of the polymer considered.

Preferably, the pendant hydrophobic groups are chosen from the linear C₂ to C₂₀ alkyls, the branched C₃ to C₂₀ alkyls, the hydrophobic amino acids, cholesterol and tocopherol.

According to a particularly preferred embodiment, the polymer according to the invention is chosen from the sodium polyglutamates and the sodium polyaspartates having a molar grafting rate with pendant hydrophobic groups ranging from 2 to 30%, the pendant hydrophobic groups being chosen from the linear C₂ to C₂₀ alkyls, the branched C₃ to C₂₀ alkyls, the hydrophobic amino acids, cholesterol and tocopherol.

According to a preferred embodiment of the invention, the polymer according to the invention has the following general structure or one of its pharmaceutically acceptable salts:

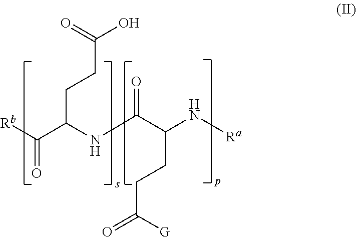

in which:
R^{a} represents a hydrogen atom, a linear C₂ to C₁₀ acyl group, a branched C₃ to C₁₀ acyl group or a pyroglutamate group.
R^{b} represents an —NHR⁵ group or an amino acid bound by the nitrogen atom the carboxyl of which is optionally substituted by an —NHR⁵ alkylamino radical or an —OR⁶ alkoxy, in which:
   R⁵ represents a hydrogen atom, a linear C₁ to C₁₀ alkyl group, a branched C₃ to C₁₀ alkyl group, or a benzyl group;
   R⁶ represents a hydrogen atom, a linear C₁ to C₁₀ alkyl group, a branched C₃ to C₁₀ alkyl group, a benzyl group or a group G;
G represents a hydrophobic group chosen from the following radicals: octyloxy-, dodecyloxy-, tetradecyloxy-, hexadecyloxy-, octadecyloxy-, 9-octadecenyloxy-, tocopheryl- and cholesteryl-, preferably alpha-tocopheryl-; a hydrophobic amino acid bound by the nitrogen atom such as leucine, valine, phenylalanine, tryptophan or tyrosine or one of their derivatives; octylamino-, dodecylamino-, tetradecylamino-, hexadecylamino-, and octadecylamino.
s corresponds to the average number of non-grafted glutamate monomers,
p corresponds to the average number of glutamate monomers bearing a hydrophobic group G,
the degree of polymerization DP=(s+p) is less than or equal to 2,000, in particular less than 700, more particularly ranging from 40 to 450, in particular from 40 to 250, and in particular from 40 to 150,

Within the meaning of the invention, general formula (II) described above covers block copolymers, but also random copolymers or multiblock copolymers.

Preferably, the sequence of the monomers of said general formula (II) is random.

According to a particularly preferred embodiment of the invention, the polymer of formula (II) has a molar grafting rate x_{P}=p/(s+p) with hydrophobic groups ranging from 2 to 30%, in particular from 4 to 15%.

Alternatively, the hydrophobic groups can be bound to the linear polymeric backbone via a spacer making it possible to bind them to the polymer chain. This spacer is advantageously divalent and belongs to the group comprising in particular the amino acid units, amino alcohol derivatives, diamine derivatives, diol derivatives and hydroxy acid derivatives. It can be for example an alanine spacer.

By way of examples, the polymer used in a composition of the invention can be a sodium polyglutamate grafted with alpha-tocopherol, a sodium polyacrylate grafted with alpha-tocopherol bound via an alanine spacer or also a poly(aspartic acid) grafted with stearyl amine.

According to a particularly preferred embodiment, the polymer used according to the invention is a polyglutamate grafted with alpha-tocopherol, in particular having a degree of polymerization ranging from 25 to 500, in particular from 40 to 150, and more particularly having a molar grafting rate with alpha-tocopherol ranging from 5 to 25%.

It can be for example a polyglutamate with a degree of polymerization of approximately 100 and having a molar grafting rate of 10% alpha-tocopherol.

Preferably, an aqueous liquid composition of the invention comprises the said polymer(s) according to the invention in a content ranging from 10 to 150 mg/mL, preferably from 30 to 70 mg/mL.

According to a variant of the composition of the invention, it comprises at least:
a sodium polyglutamate polymer with a degree of polymerization ranging from 40 to 150, in particular approximately 100 and grafted from 5 to 25% with alpha-tocopherol groups, preferably approximately 10%; and
a prodrug of buprenorphine or an analogue of buprenorphine, in particular a prodrug of buprenorphine chosen from buprenorphine-isobutyrate and buprenorphine-enanthate.

Within the context of this embodiment variant, the prodrug(s) of buprenorphine or an analogue of buprenorphine and the said sodium polyglutamate(s) can advantageously be present in a composition of the invention in a prodrug(s)/polymer(s) ratio by weight ranging from 0.02 to 0.2.

According to a particular embodiment, a composition according to the invention can contain as active ingredient, as well as the prodrug of buprenorphine, unmodified buprenorphine or analogue of buprenorphine.

By “unmodified” is meant in particular that buprenorphine or the analogue of buprenorphine is not in the form of a prodrug.

Preferably, the unmodified buprenorphine is in a water-soluble form in particular in the form of its hydrochloride (BuHCl).

Such a combination is advantageous in order to provide jointly an immediate and differed analgesic effect. This combination is particularly welcome in the case of a post-surgical administration, where it is desirable to obtain the analgesic effect rapidly and to maintain this effect over a period of several days.

Preferably, the molar ratio of unmodified buprenorphine or analogue of buprenorphine to the prodrug of buprenorphine or analogue of buprenorphine is comprised between 0.1 and 2.

Advantageously, the polymers of the invention are dispersible in water in the state of nanoparticles or nanogels having a size less than 200 nm.

The corresponding compositions according to the invention are therefore sterilizable by filtration, in particular by filtration on membranes of approximately 0.2 μm.

The average size of the nanoparticles or nanogels can be measured by quasi-elastic light scattering, by techniques known to a person skilled in the art.

The formulations of the invention can be more particularly selected in such a way that they occur in the form of a dispersion of nanoparticles or nanogels having an average size of less than 200 nm, preferably less than 100 nm.

Preparation Method:

Generally speaking, the compositions according to the invention are prepared by solubilizing the prodrug in a solution of the specific polymer considered.

The solubilization of the prodrugs in the solution of polymer can be carried out directly by adding the desired quantities and by leaving under stirring at room temperature. Techniques such as heating, using ultrasound or using an organic solvent can also be used to facilitate the solubilization. In cases where an organic solvent is used, it can be removed by dialysis if necessary.

According to an embodiment variant, the prodrug of buprenorphine is solubilized in an aqueous solution of the polymer and the insoluble part is separated out by filtration or centrifugation. An aqueous solution is obtained the pH of which can be adjusted to between 5 and 8 by adding acetic acid, soda or hydrochloric acid.

According to another embodiment variant, the method according to the invention can then subsequently comprise a stage of dehydrating the suspension of the obtained particles in order to obtain them in the form of a dry powder.

As specified above, the present invention relates to, according to yet another of its aspects, a solid composition comprising at least one prodrug with low aqueous solubility of said buprenorphine or an analogue of buprenorphine and at least one polymer with a linear backbone chosen from the polyglutamates, polyaspartates, poly(meth)acrylates and polysaccharides, and to which one or more hydrophobic groups are grafted.

Such a solid composition, generally in the form of a powder, can be in particular obtained by dehydration of an aqueous liquid composition as described previously.

The aqueous liquid composition can be dehydrated by a standard method of dehydration such as lyophilization, atomization or evaporation, preferably by lyophilization.

In this embodiment variant, the aqueous liquid composition according to the invention can thus be reconstituted, before its use, by the simple addition of an aqueous liquid, in particular water, to the powder of the invention, and optionally by manual stirring using appropriate means.

The aqueous liquid can be more particularly composed of water, in particular from water for injection, also called water “for injectable preparations” or “sterile water for injection”, which is water which is sterile and free from pyrogen.

Advantageously, the stages of dehydration and reconstitution of an aqueous liquid solution according to the invention in no way affects the pharmacokinetic properties of the composition obtained, in particular with regard to the controlled release of buprenorphine or analogue of buprenorphine.

The composition according to the invention can be administered by oral, pulmonary, parenteral, nasal, vaginal, ocular, subcutaneous, intravenous, intramuscular, intradermal, intraperitoneal, intracerebral or buccal route.

Preferably, the composition is administered by parenteral route, in particular by subcutaneous or intramuscular route.

According to another embodiment, the composition can optionally contain at least one excipient known to a person skilled in the art, in particular chosen from the excipients usually used for adjusting the pH and the osmolarity, in order to improve the stability (antioxidant), as an antimicrobial agent or as a lyoprotectant.

These excipients are well known to a person skilled in the art (for example described in the work: Injectable Drug Development, P. K. Gupta et al. Interpharm Press, Denver, Colo. 1999). For example, the formulation can contain antimicrobial agents such as benzyl alcohol, phenol or metacresol at concentrations of a few percent, the optimum of which is determined experimentally.

The invention will be better explained by the examples hereafter, given only by way of illustration.

FIGURES

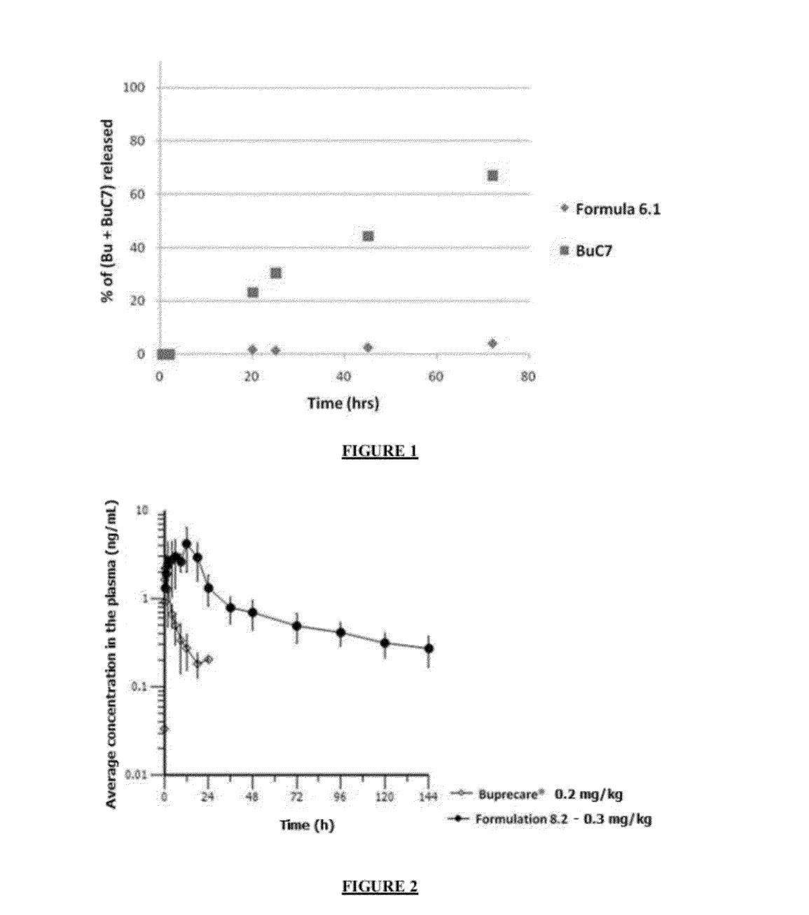

FIG. 1: in vitro release profiles of buprenorphine BuC7 in its solid form (▪ BuC7) and in its solubilized form in a solution of the polymer PGAT-1 (♦ Formula 6.1).

FIG. 2: average concentration profiles in the plasma of buprenorphine after injection of a formulation according to the invention (-•- Formulation) and a commercial formulation (-o- Buprecare®).

### EXAMPLES

### Example 1

### Synthesis of buprenorphine 6-isobutyrate (BuC4)

The synthesis of buprenorphine 6-isobutyrate is carried out by reaction of isobutyl chloride (1.1 eq.) with buprenorphine (1.0 eq.) in the presence of triethylamine (3.0 eq.) in dichloromethane (0.07 mmol buprenorphine/mL). The reaction is followed by thin layer chromatography (Silica plates F254, eluent ethyl acetate/heptane 2/8 (v/v), phosphomolybdic acid heat development). The reaction is complete (total conversion of the starting product) after approximately 10 min. The product is isolated by washes of the organic phase, drying and evaporation of the solvent under vacuum. 1.0 g of synthesized product is obtained. The structure of the product is confirmed by ¹H/¹³C₁D and 2D NMR.

### Example 2

### Synthesis of buprenorphine 6-enanthate (BuC7)

The synthesis of buprenorphine 6-enanthate is carried out by reaction of heptanoyl chloride (1.1 eq.) with buprenorphine (1.0 eq.) in the presence of triethylamine (3.0 eq.) in dichloromethane (0.09 mmol buprenorphine/mL). The reaction is followed by thin layer chromatography (Silica plates F254, eluent ethyl acetate/heptane 5/5 (v/v), phosphomolybdic acid heat development). The reaction is complete (total conversion of the starting product) after a few hours. The product is isolated by washes of the organic phase, drying and evaporation of the solvent under vacuum. 2.6 g of synthesized product is obtained. The structure of the product is confirmed by ¹H/¹³C 1D and 2D NMR.

### Example 3

### Synthesis of buprenorphine 6-myristate (BuC14)

The synthesis of buprenorphine 6-myristate is carried out by the reaction of myristoyl chloride (1.1 eq.) with buprenorphine (1.0 eq.) in the presence of triethylamine (3.0 eq.) in dichloromethane (0.04 mmol buprenorphine/mL). The reaction is followed by thin layer chromatography (Silica plates F254, eluent ethyl acetate/heptane 2/8 (v/v), phosphomolybdic acid heat development). The reaction is complete (total conversion of the starting product) after a few hours. The product is isolated by washes of the organic phase, drying and evaporation of the solvent under vacuum. 1.0 g of synthesized product is obtained. The structure of the product is confirmed by ¹H/¹³C 1D and 2D NMR.

### Example 4

### Synthesis of Amphiphilic Polymers According to the Invention

4.1. Synthesis of sodium poly(L-glutamate) polymers with a Degree of Polymerization of Approximately 100 and Grafted at 10 mol % (Called PGAT-1 in the Following) or 20 mol % (PGAT-2) in Alpha-Tocopherol

The sodium poly(L-glutamate) polymers with a degree of polymerization of approximately 100 and containing 10 mol % (PGAT-1, molar mass 19 KDa) or 20 mol % (PGAT-2, molar mass 23 KDa) of alpha-tocopherol (all-racemic) are synthesized according to the operating method described in WO 03/104303. The size of the particles, measured by light scattering is comprised between 10 and 20 nm for both polymers.

### 4.2. Synthesis of a Sodium Polyacrylate Polymer with a Molar Mass of Approximately 34 KDa (in PMMA equivalent) and Grafted at 5 mol % in Alpha-Tocopherol Bound Via an Alanine Spacer (PAAT-1)

The polymer PAAT-1 is obtained in a similar fashion by reacting an alanine derivative of tocopherol with acrylic acid.

Stage 1: Purification of the Commercial Poly(Acrylic Acid) (Degacryl 4779L):

75 g of DEGACRYL 4779L solution (sold by Evonik) are diluted with 1.425 g of milli-Q water then diafiltered against 8 volumes of water. The solution obtained is then lyophilized. The average molecular mass Mn, measured by size exclusion chromatography, is 33.6 KDa in PMMA (polymethyl methacrylate) equivalent and the polydispersity index is 2.4.

Stage 2: Synthesis of Alpha-Tocopheryl Alaninate (AlaVE)

22.08 mL of N,N′-Diisopropylcarbodiimide (DIPC) is added to a solution of 21.1 g of N-Boc L-alanine, 40 g of alpha-tocopherol (all-racemic) and 0.567 g of dimethylaminopyridine (DMAP) in 400 mL of dichloromethane. After stirring at 20° C. for 22 hours, the reaction mixture is successively washed with a solution of 0.1 N HCl, water, a 5% sodium bicarbonate solution and finally water. The organic phase is evaporated to dryness and the oil obtained is solubilized in 400 mL of a 4M HCl solution in dioxane. After stirring for 4 hours at room temperature, the reaction mixture is evaporated to dryness and crystallized from ethanol. AlaVE hydrochloride (33.8 g of a white powder) thus prepared is analyzed by proton NMR in CDCl₃ and exhibits a spectrum in accordance with its chemical structure.

Stage 3: Grafting of the AlaVE on the Purified Poly(Acrylic Acid):

3.74 g of AlaVE is solubilized in 50 mL of DMF and 0.97 mL of triethylamine. In parallel, 10 g of purified Degacryl (stage 1) are dissolved in 250 mL of N,N-dimethylformamide (DMF) and 0.34 g of 4-dimethylaminopyridine (DMAP). This solution is cooled down to 15° C., and the suspension of AlaVE/triethylamine then 1.93 g of N,N′-Diisopropylcarbodiimide (DIPC) are added successively. The reaction mixture is stirred overnight at 15° C. After the addition of a 35% HCl solution (1.16 mL) diluted in 3 mL of DMF, the reaction mixture is neutralized with 1 N soda in 900 mL of water. The solution obtained is diafiltered against 8 volumes of salt water (0.9% NaCl), then 4 volumes of water, and concentrated until obtaining a volume of approximately 400 mL. 100 mL of ethanol is added, and the solution obtained is stirred overnight at room temperature, then diafiltered against 8 volumes of water and concentrated up to a concentration of approximately 45 g/L.

The percentage of grafted AlaVE, determined by proton NMR in TFA-d, is 5.3%. The average size of the particles, measured by light scattering, is 17 nm. The average molar mass is 37 KDa (PMMA equivalents) and the polydispersity index is 2.6. The viscosity of the polymer at 30 mg/ml is 6 mPa·s at 10 s⁻¹.

4.3. Synthesis of a Polyaspartic Acid Polymer with a Molar Mass of Approximately 9 KDa (in PMMA Equivalents) and Grafted at 20% in Octadecylamine.

A polyaspartic acid polymer grafted with 20% of stearylamine (PAspC-1) was prepared according to the two stages below.

Stage 1: Synthesis of Polysuccinimide by Polycondensation of Aspartic Acid

The polysuccinimide was synthesized according to a protocol similar to that described in Polymer 1997, 38(18), 4733-4736 using L-aspartic acid.

50 g of aspartic acid is introduced into 260 mL of mesitylene and 40 mL of sulpholane. 0.1 equivalent of orthophosphoric acid is added and the mixture thus obtained is heated at reflux for 8 hours. The suspension is then filtered on a frit; the solid recovered is rinsed with ethanol, with water then again with ethanol and dried under vacuum at 80° C. overnight. The polysuccinimide intermediate is obtained in the form of a beige powder.

Stage 2: Aminolysis with Stearylamine then Hydrolysis of the Residual Polysuccinimide Groups.

The protocol is similar to that described in Langmuir 2001, 17, 7501.

5 g of this intermediate is introduced into 30 mL of DMF. 0.2 equivalent (with respect to the succinimide monomer unit) of stearylamine is added. The mixture is heated at 80° C. overnight. The crude reaction product is poured into an aqueous 1 N soda solution. The milky suspension obtained is stirred for a few hours at room temperature, then poured into an excess of methanol. The expected polymer precipitates. It is isolated by filtration on a frit, rinsing and drying under vacuum.

The product is redissolved in water while heating at 80° C. under magnetic stirring for 1 h, at a concentration of 10 mg/mL. Purification is carried out by diafiltration against water. The polymer is then concentrated to approximately 60 mg/g and obtained in the form of a clear solution.

The grafting rate of 20% is determined by ¹H NMR in TFA-d. An average molar mass by weight of 8,900 Da is determined by size exclusion chromatography using a PMMA calibration. Objects with an average volume diameter of approximately 10 nm are obtained (measured by DLS). The viscosity of a solution of this polymer at a concentration of 58 mg/g is 10 mPa·s at 10 s⁻¹.

All these polymers are soluble in water at neutral pH or pH close to neutrality, form nanogels having an average size less than 100 nm and can be filtered through a 0.22 μm filter.

### Example 5

### Solubilization of the Prodrugs in a PBS Buffer

Table 1 below shows the solubilities of the prodrugs of buprenorphine and of buprenorphine hydrochloride (BuHCl), determined in a PBS buffer (containing 137 mM of NaCl (8.0 g/L), 2.7 mM of KCl (0.2 g/L), 10 mM of Na₂HPO₄ (1.44 g/L) and 1.76 mM of KH₂PO₄ (0.24 g/L)), by the additions of powder in a given volume of buffer solution and observing the clearness by eye.

TABLE 1

| No. | Formula | pH | Amount of solubilized prodrug | |
|---|---|---|---|---|
| 5.1 | BuC4 | 7 | <0.8 | mg/mL |
| 5.2 | BuC7 | 7 | <0.6 | mg/mL |
| 5.3 | BuHCl | 7 | 17 | mg/mL * |

* AAPS PharmSciTech 2007; 8 (3) Article 62

It is observed that the prodrugs' solubilities are significantly lower than 1 mg/mL of buffer.

### Example 6

### Solubilization of the Prodrugs in a Suspension of Amphiphilic Polymer

Table 2 below shows, as examples of solubilization in solutions of amphiphilic polymers of the invention synthesized in the previous Example 4, the prototypes of formulations produced.

TABLE 2

| | | | Polymer | | | Amount of solubilized |
|---|---|---|---|---|---|---|
| No. | Prodrug | Polymer | concentration | | pH | prodrug |
| 6.1 | BuC7 | PGAT-1 | 43 | mg/mL | 6 | 2.3 mg/mL |
| 6.2 | BuC7 | PGAT-1 | 43 | mg/mL | 7 | 3.1 mg/mL |
| 6.3 | BuC7 | PGAT-2 | 90 | mg/mL | 7 | 1.1 mg/mL |
| 6.4 | BuC7 | PAspC-1 | 68 | mg/g | 7 | 1.0 mg/mL |
| 6.5 | BuC4 | PAspC-1 | 68 | mg/g | 7 | 2.4 mg/mL |

It is noted that the solubility of the prodrugs is improved by the presence of amphiphilic polymers.

### Example 7

### Comparison of the Solubilization of the Prodrugs BuC14 in a Solution of Sodium Polyacrylate Grafted at 6 Mol % in Alpha-Tocopherol Bound Via an Alanine Spacer and in Water

One sample containing 0.4 mg/mL of BuC14 is prepared in an aqueous solution having a concentration in sodium polyacrylate grafted at 6 mol % in alpha-tocopherol bound via an alanine spacer of 32.4 mg/mL.

After stirring for 24 hours at room temperature, a visual control shows that the BuC14 sample in the polymer solution is perfectly clear. Thus, the amount of prodrug solubilized in the polymer solution is 0.4 mg/mL.

As a reference, one sample containing 0.2 mg/mL of BuC14 is prepared in water.

After stirring for 24 hours at room temperature, some undissolved product remains in the BuC14 sample. Thus, the amount of prodrug solubilized in water is less than 0.2 mg/mL.

This shows that the prodrug solubility is increased in the presence of the amphiphilic polymer.

### Example 8

### In Vitro Release of the Prodrugs

2 mL of formulation 6.1 of Example 6 is introduced into dialysis tubing of 1 kDa closed at both ends. Then the tubing is placed in 500 mL of water containing 0.1% of acetic acid maintained at 37° C. and under mechanical stirring at 100 rpm. At regular time intervals, samples of the aqueous phase are taken and the quantity of prodrug released is assayed. This assay measures the amount of buprenorphine both under BuC7 form and Bu form.

For the prodrug alone, the solubilization rate of 4 mg of powder in 500 mL of 0.1% acetic acid solution is measured.

FIG. 1 shows that the prodrug BuC7 alone is solubilized over time and that for the formulation 6.1, there is no or very little release of the prodrug.

This shows that BuC7 prodrug in formulation 6.1 remained largely associated to the amphiphilic polymer, inside the dialysis tubing.

### Example 9

### Preparation and Characterization of the Formulations which can be Used for the Treatment of Pain

Without it being limitative, Table 3 below describes formulations which can be injected into humans or animals. They are prepared by solubilizing the prodrug in an aqueous solution of the polymer (PGAT-1 at 45 mg/mL) and the osmolality is adjusted using sucrose and the pH is adjusted using an acetic acid/sodium acetate buffer. The formulation obtained is rendered sterile by filtration on a 0.2 μm filter. The quantity of prodrug or active ingredient is measured by HPLC.

The data relating to these formulations are shown in Table 3 below.

TABLE 3

| | Concentration | | | Osmolality | Diameter | Viscosity (mPa · s |
|---|---|---|---|---|---|---|
| No. | (mg/mL) | Appearance | pH | (mOsm/kg) | (nm) | at 10 s⁻¹) |
| 9.1 | 2.4 BuC4 | Clear | 6 | 303 | 12 | 5 |
| 9.2 | 2.3 BuC7 | Clear | 6 | 338 | 12 | 5 |
| 9.3 | 0.4 BuHCl | Clear | 6 | 331 | 11 | 4 |
| | 1.8 BuC7 | | | | | |

All the formulations are clear and no precipitation or settlement is observed after 3 months at 4° C. They are easily injectable through a 27 G or 30 G needle.

As a counter-example, a control formulation was prepared from the mixture of active ingredients considered in test 9.3, namely BuHCl and BuC7 in sesame oil, a medium which is favourable to the solubilization of BuC7. It appears that this same mixture of active ingredients cannot be solubilized in it and that its injection requires 18 to 21 G needles in view of its too high viscosity.

The aqueous solution complemented with amphiphilic polymer considered according to the invention therefore allows this problem to be effectively overcome and moreover allows an adjustment of the active ingredient/prodrug ratio in order to obtain both a rapid and sustained release profile of the analgesic active principle.

### Example 10

### Pharmacokinetics of Buprenorphine in the Dog after the Subcutaneous Injection of Formulation 9.2 (Example 9) of Buprenorphine 6-Enanthate in an Aqueous Solution of PGAT-1

Formulation 9.2 of Example 9 of buprenorphine 6-enanthate (equivalent buprenorphine base concentration of 2 mg/mL) is injected by subcutaneous route into Beagle dogs (n=4) at a dose of 0.3 mg/kg (buprenorphine base equivalent). Blood samples are taken just before the treatment and then at 0.5, 1, 2, 4, 6, 9, 12, 18, 24, 36, 48, 72, 96, 120 and 144 hours after the administration. In parallel the commercial formulation for immediate release (Buprecare®) is administered at a dose of 0.02 mg/kg to another group of Beagle dogs (n=4). Blood samples are taken before the treatment and then at 0.125, 0.25, 0.5, 1, 2, 4, 6, 9, 12, 18 and 24 hours after the administration.

The concentration of buprenorphine in the plasma is measured in these samples by LC-MS/MS (HPLC system 1100 Series from Agilent with an XTerra MS C18 column, 3.5 μm; 2.1×150 mm from Waters, and an API 3000 mass spectrometry detector from Applied Biosystem).

The average concentration profile in the plasma shown in FIG. 2 demonstrates the sustained release of buprenorphine in the plasma in comparison to a commercial formulation (Buprecare®) administered at a lower dose. The extension of the release of buprenorphine is estimated by the parameters:
Tₘₐₓ: median of the time at which the concentration in the plasma is at a maximum,
Cₘₐₓ/D: average of the maximum concentrations relative to the dose D,
T_{50% AUC}: time at the end of which 50% of the AUC is reached calculated over the interval of measurement of the concentrations.

In this example, the time Tₘₐₓ and T_{50% AUC} are 12 h and 22 h for the formulation 9.2 against 0.75 h and 4 h for the commercial formulation. This shows the extension of the release in the case of a formulation according to the invention.

The parameters Cₘₐₓ/D are equal to 18 ng/mL/(mg/kg) for formulation 8.2 against 114 ng/mL/(mg/kg) for the commercial formulation, which shows the reduction in Cₘₐₓ/D associated with the extension of the release of buprenorphine.

TABLE 4

| | Dose | Cₘₐₓ | Tₘₐₓ | | Cₘₐₓ/D | T_{50% AUC} |
|---|---|---|---|---|---|---|
| Formulation | (mg/kg) | (ng/mL) | (h) | | (ng/mL/(mg/kg)) | (h) |
| Buprecare ® | 0.02 | 2.3 ± 1.0 | 0.75 | (0.5-1) | 114 ± 48 | 4 ± 2 |
| Formulation 8.2 | 0.3 | 5.4 ± 1.1 | 12 | (6-18) | 18 ± 4 | 22 ± 8 |

It is demonstrated that the formulation of commercial buprenorphine hydrochloride has rapid pharmacokinetics with a half-life of the order of 12 hours while the formulation of polymer containing the prodrug BuC7 has an apparent half-life of the order of 66 hours in terms of buprenorphine released. It is noted that the concentration of buprenorphine in the plasma is greater than 0.3 ng/mL up to 120 hours, which should allow the analgesic effect to be maintained throughout this period. Moreover, it is observed that buprenorphine rapidly appears in the plasma with a concentration greater than or equal to 0.5 ng/mL after only one hour.

### Example 11

### Preparation of a Formulation of Buprenorphine 6-Enanthate in an Aqueous Solution of PGAT-1 in a Freeze-Dried Form and Stability Study

Formulation 11 is prepared by solubilizing the BuC7 prodrug in an aqueous solution of the polymer (PGAT-1 at 45 mg/mL) similarly to the operating method described in Example 9 (formulation 9.2). The formulation is then freeze-dried and stored under solid form at 4° C. or 25° C. The stability of the freeze-dried formulation is measured by HPLC (measure of the BuC7 prodrug amount and buprenorphine amount), after reconstitution by adding the suitable amount of water and stirring a few hours at room temperature.

The data relating to the stability of the freeze-dried formulation after 6 months at 4° C. and 6 months at 25° C. are shown in Table 5 below.

TABLE 5

| Storage | Storage | BuC7 prodrug amount | Buprenorphine amount |
|---|---|---|---|
| time | conditions | (mg/g of formulation) | (mg/g of formulation) |
| 0 months | — | 2.4 | 0.1 |
| 3 months | 4° C. | 2.3 | 0.1 |
| | 25° C. | 2.3 | 0.1 |
| 6 months | 4° C. | 2.3 | 0.1 |
| | 25° C. | 2.2 | 0.2 |

The freeze-dried formulation thus prepared shows a good stability after 6 months.

1. Aqueous liquid pharmaceutical composition, for the controlled release of buprenorphine or an analogue of buprenorphine, comprising at least one prodrug with low aqueous solubility of said buprenorphine or of an analogue of said buprenorphine and at least one polymer having a linear backbone chosen from the polyglutamates, polyaspartates, poly(meth)acrylates and polysaccharides, to which one or more hydrophobic groups are grafted.

2. Composition according to claim 1, characterized in that the chemical formula of said prodrug of buprenorphine or an analogue of buprenorphine comprises at least the following structural unit: 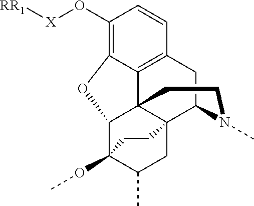
in which R is chosen from the groups:
linear C₂ to C₂₀ alkyls, branched C₃ to C₂₀ alkyls optionally comprising at least one unsaturation,
C₃ to C₆ cyclic alkyls; and
substituted phenyl and phenalkyls;
X is a C═O, O—C═O or NH—C═O group; and
the dotted lines show the potential substitution sites.

3. Composition according to claim 1 or 2, characterized in that the prodrug of buprenorphine corresponds to the following general formula (I): 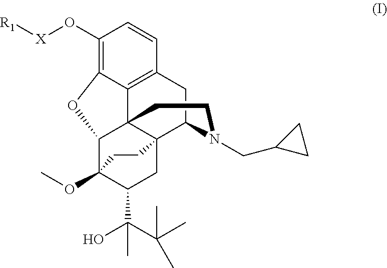
in which R and X are as defined in claim 2.

4. Composition according to claim 2 or 3, characterized in that R is chosen from the ethyl, propyl, iso-propyl, butyl, iso-butyl, sec-butyl, hexyl, 2-ethylhexyl, cyclohexyl, heptyl, octyl, dodecyl, tridecyl, tetradecyl, pentadecyl, hexadecyl, heptadecyl, octadecyl, phenyl and benzyl groups.

5. Composition according to any one of the previous claims, characterized in that the prodrug is a prodrug of ester type of said buprenorphine, in particular of formula (I) as defined in claim 3 in which X is a C═O group, and is more particularly chosen from buprenorphine 6-isobutyrate, buprenorphine 6-enanthate and buprenorphine 6-myristate.

6. Composition according to any one of the previous claims, characterized in that it comprises from 0.5 mg/mL to 10 mg/mL, in particular from 1 to 5 mg/mL, of prodrug(s) of buprenorphine or an analogue of buprenorphine.

7. Composition according to any one of the previous claims, characterized in that said polymer is chosen from the sodium polyglutamates and sodium polyaspartates, in particular having a molar grafting rate with pendant hydrophobic groups ranging from 2 to 30%, said pendant hydrophobic groups being chosen from the group comprising the linear or branched C₂ to C₂₀ alkyls, the hydrophobic amino acids, cholesterol and tocopherol.

8. Composition according to any one of the previous claims, characterized in that said polymer has the following general structure, or one of its pharmaceutically acceptable salts: 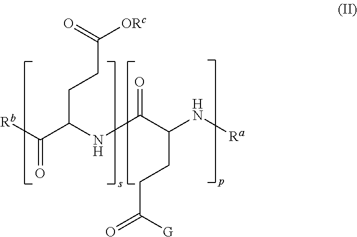
in which:
R^{a} represents a hydrogen atom, a linear C₂ to C₁₀ acyl group, a branched C₃ to C₁₀ acyl group or a pyroglutamate group.
R^{b} represents an —NHR⁵ group or an amino acid bound by the nitrogen atom the carboxyl of which is optionally substituted by an —NHR⁵ alkylamino radical or an —OR⁶ alkoxy, in which:
R⁵ represents a hydrogen atom, a linear C₁ to C₁₀ alkyl group, a branched C₃ to C₁₀ alkyl group, or a benzyl group;
R⁶ represents a hydrogen atom, a linear C₁ to C₁₀ alkyl group, a branched C₃ to C₁₀ alkyl group, a benzyl group or a group G;
R^{c} represents a hydrogen atom or a monovalent metallic cation, preferably a sodium or potassium ion,
G represents a hydrophobic group chosen from the following radicals: octyloxy-, dodecyloxy-, tetradecyloxy-, hexadecyloxy-, octadecyloxy-, 9-octadecenyloxy-, tocopheryl- and cholesteryl-, preferably alpha-tocopheryl-; a hydrophobic amino acid bound by the nitrogen atom such as leucine, valine, phenylalanine, tryptophan or tyrosine or one of their derivatives; octylamino-, dodecylamino-, tetradecylamino-, hexadecylamino-, and octadecylamino.
s corresponds to the average number of non-grafted glutamate monomers,
p corresponds to the average number of glutamate monomers bearing a hydrophobic group G,
the degree of polymerization DP=(s+p) is less than or equal to 2,000, in particular less than 700, more particularly ranging from 40 to 450, in particular from 40 to 250, and in particular from 40 to 150.

9. Composition according to any one of the previous claims, characterized in that said polymer is a polyglutamate grafted with alpha-tocopherol, in particular having a degree of polymerization ranging from 25 to 500, in particular from 40 to 150, and more particularly having a molar grafting rate with alpha-tocopherol ranging from 5 to 25%.

10. Composition according to any one of the previous claims, characterized in that it further contains unmodified buprenorphine or analogue of buprenorphine in a water-soluble form, in particular buprenorphine in the form of its hydrochloride.

11. Composition according to claim 10, characterized in that the ratio of unmodified buprenorphine or analogue of buprenorphine to the prodrug of buprenorphine or analogue of buprenorphine is comprised between 0.1 and 2.

12. Composition according to any one of the previous claims, characterized in that it occurs in the form of a dispersion of nanoparticles or nanogels having an average size of less than 200 nm, preferably less than 100 nm.

13. Composition according to any one of the previous claims, characterized in that it has a viscosity, measured at 20° C. and at a shear rate of 10 s⁻¹, of less than 200 mPa·s, preferably comprised between 2 and 100 mPa·s.

14. Composition according to any one of the previous claims, characterized in that it is obtained by the addition of an aqueous liquid to a composition in the form of a powder having been formed beforehand by dehydration of an aqueous liquid composition as defined according to any one of claims 1 to 13.

15. Solid composition in the form of a powder obtained by dehydration of an aqueous liquid composition as defined according to any one of claims 1 to 14.
